(19) United States
(12) Patent Application Publication
(10) Pub. No.: US 2009/0297612 A1
(43) Pub. Date: Dec. 3, 2009

(54) HOMOGENEOUS, INTRINSIC RADIOPAQUE EMBOLIC PARTICLES

(75) Inventors: Levinus Hendrik Koole, Gulpen (NL); Catharina Sibilla Josephine Van Hooy-Corstjens, Roosteren (NL)

Correspondence Address: BANNER & WITCOFF, LTD., 28 STATE STREET, SUITE 1800, BOSTON, MA, 02109-1701, US

(73) Assignees: Universiteit Maastricht, Maastricht (NL); Stichting voor de Technische Wetenschappen, Utrecht (NL)

(21) Appl. No.: 12/432,457

(22) Filed: Apr. 29, 2009

Related U.S. Application Data
(63) Continuation of application No. PCT/NL2007/050522, filed on Oct. 31, 2007.

(30) Foreign Application Priority Data
Oct. 31, 2006 (EP) 06076955.1

(51) Int. Cl. A61K 31/78; A61K 9/14; A61P 35/00; A61P 7/00; A61P 7/04

(52) U.S. Cl. 424/489; 424/78.31

(57) ABSTRACT

The invention is directed to embolic material comprising spherical, homogeneous and substantially non-porous radiopaque polymer particles based on at least one hydrophilic monomer and at least one radiopaque monomer according to general formula

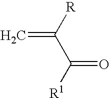

wherein R is H, methyl or ethyl, and R¹ is I, Br or

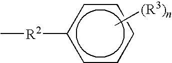

wherein R² is O, NH, O—[CH₂—CH₂—O]ₚ—C(O)—, O—[CH₂]ₘ—O—C(O)—, O—[CH₂]ₚ—, NH—[CH₂—CH₂—O]ₚ—C(O)—, NH—[CH₂]ₘ—O—C(O)- or NH—[CH₂]ₚ— wherein m>1 and p≧1, R³ is I or Br and n is 1, 2 or 3,
the iodine and/or bromine content being at least 5 wt. % based on the dry weight of the particle, the said particles having an average particle diameter of at least 10 μm and being able to imbibe water up to a volume increase of the particle of at least 10%.

### RELATED APPLICATIONS

This application is a continuation of PCT application no. PCT/NL2007/050522, designating the United States and filed Oct. 31, 2007; which claims the benefit of the filing date of European application no. 06076955.1 filed Oct. 31, 2006; each of which is hereby incorporated herein by reference in its entirety for all purposes.

### FIELD

The invention is directed to solid, homogeneous radiopaque copolymer particles, with controllable swelling properties, and the use thereof in embolisation.

### BACKGROUND

Artificial emboli are intensively used by interventional radiologists in minimally invasive procedures to achieve vascular occlusion. Embolisation therapy may be utilised to assist in the management of arteriovenous malformations, fibroids, neoplasms, definitive treatment of tumors (usually benign), for palliative embolisation and for preoperative embolisation. For example, the preferred treatment of arteriovenous malformations larger than 3 cm consists of two steps: (i) embolisation, triggering a size reduction of 10-95%, and (ii) subsequent microsurgical resection or stereotactic surgery. Another example is found in the treatment of intracranial meningiomas. While microsurgical removal is still the treatment of choice, it has become clear that superselective embolisation can lead to significant shrinkage of the tumor. Therefore, embolisation is an attractive alternative to microsurgery, especially for critically ill people, where microsurgery is equivocal.

Commercial embolic agents for vascular occlusion include fluids, mechanical devices and particles. The choice for a specific material depends on many factors, such as the type of lesion to be treated and the kind of catheter to be used. Particles for embolisation mainly comprise polymers, both natural and synthetic. Polymeric embolic agents have an advantage in their good biocompatibility towards patients' tissues, they are able to keep the formed thrombus and are encapsulated very fast.

An important shortcoming of polymeric embolic particles that have been applied so far is that they are radiolucent, i.e. they are invisible on X-ray images. Consequently, complications such as ‘reflux with non-target embolisation’ and ‘through embolisation’ are essentially undetectable. To deal with this problem, the embolic particles are usually dispersed in saline which has been enriched with contrast medium. This has the disadvantage that fluoroscopic exploration, which is performed during injection of the emboli through a catheter, only provides information about the location of the fluid and not about the embolic particles themselves. In case of improper dispersion of the particles, it is well possible that the liquid can pass more distal in the tumor than the particles, so improper location of the embolic agent is inferred from this method. To verify the exact location of the embolic particles, it would therefore be advantageous to have a polymeric particulate embolic agent that is radio-opaque.

Radio-opaque polymeric particles are described in U.S. Pat. No. 4,622,367. The particles contain a derivative of an amino-triiodobenzoic acid. The radiopaque particles are obtained by swelling hydrogel particles, that are based on polymers and copolymers of acrylates and methacrylates and contain hydroxyl or epoxide groups on side chains of the polymer skeleton, in an excess of a solvent which contains a dissolved derivative of amino-triiodobenzoic acid. The method thus involves at least two steps. Further, the derivative of amino-triiodobenzoic acid has to diffuse into the hydrogel particles.

Due to the large size of the molecule, the diffusion of the derivative of the amino-triiodobenzoic acid will be limited and consequently the derivative will mainly be present at the outer parts of the spheres. This results in a non-homogeneous, core-shell type structure of the sphere. Since this derivative is hydrophobic in character, high concentrations of this compound at the outer surface of the sphere will extremely limit the water transport inside the sphere and consequently the material will lose its hydrophilic character and consequently also the swelling properties in water.

Horák el al. (D. Horák, M. Metalová, F. Rypá\check{c}ek J. Biomed. Mater. Res. 1996, 34(2), 183-188) describe also radiopaque particles. The particles are prepared by radical suspension copolymerisation of 2-hydroxyethyl methacrylate, 3-(methacryloylamidoacetamido)-2,4,6-triiodobenzoic acid and ethylene dimethacrylate in an aqueous medium and in the presence of large amounts of organic solvent, acting as template for the porosity. Because the particles are very porous, high amounts of iodine are required to render the particles sufficiently radio-opaque for use in embolisation. This also diminishes the hydrophilic character of the material.

Object of the present invention is to overcome one or more of these disadvantages of the prior art. This object has been achieved by providing radiopaque copolymer particles based on an iodine or bromine substituted radiopaque monomer having specific properties as to hydrophilicity, opacity and particle size.

The foregoing and other features and advantages of the present invention will be more fully understood from the following detailed description of illustrative embodiments taken in conjunction with the accompanying drawings.

### BRIEF DESCRIPTION OF THE DRAWINGS

FIGS. 1A-1C depict x-ray images of radiopaque microspheres. (A) Single spheres, recorded at clinical conditions. (B) 10 mg of spheres in a chicken leg, recorded at clinical conditions. (C) 10 mg of spheres in a chicken leg, recorded at clinical conditions.

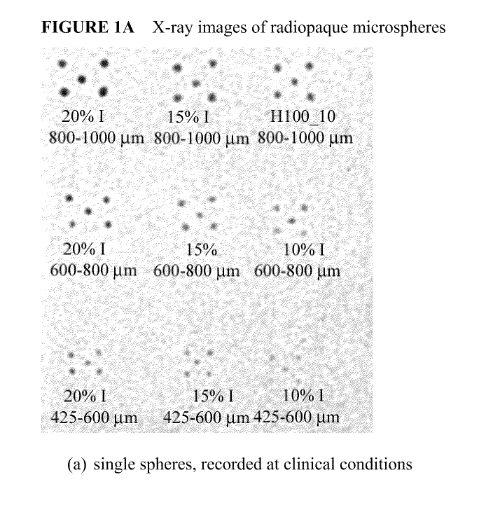

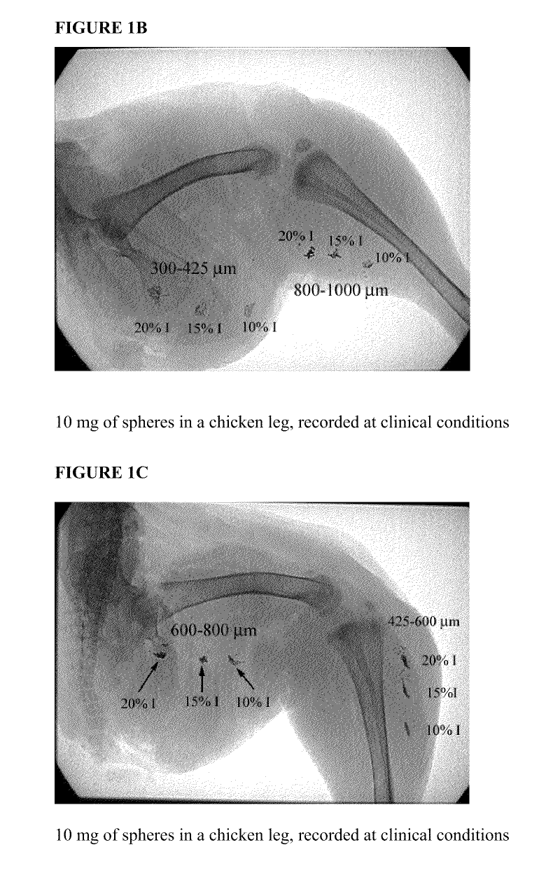

### DETAILED DESCRIPTION

Accordingly, the present invention is directed embolic material comprising spherical, homogeneous and substantially non-porous radiopaque polymer particles based on at least one hydrophilic monomer and at least one radiopaque monomer according to general formula

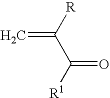

wherein R is H, methyl or ethyl, and R¹ is I, Br or

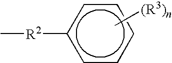

wherein R² is O, NH, O—[CH₂—CH₂—O]ₚ—C(O)—, O—[CH₂]ₘ—O—C(O)—, O—[CH₂]ₚ—, NH—[CH₂—CH₂—O]ₚ—C(O)—, NH—[CH₂]ₘ—O—C(O)— or NH—[CH₂]ₚ— wherein m>1 and p≦1, R³ is I or Br and n is 1, 2 or 3, the iodine and/or bromine content being at least 5 wt. % based on the dry weight of the particle, the said particles having an average particle diameter of at least 10 μm and being able to imbibe water up to a volume increase of the particle of at least 10%.

Methods for making such monomeric compounds are for example disclosed in WO-A-96/05872. Preferably m or p are below 10. Preferably m is 2. Preferably p is 1 or 2.

R³ can be located at all possible positions, being ortho, meta, and para. In case n is 1, R³ is preferably located at position 2 or 4. Most preferably at position 4. In case n is 2, R³ can be located at position 2 and 4 (ortho and para respectively) or position 3 and 5 (meta). In case n is 3, R³ is preferably located at positions 2, 3 and 5.

Preferably, a monomer comprising covalently bound iodine is used. Examples of suitable radio-opaque monomers are 2-[2′-iodobenzoyl]-oxo-ethyl methacrylate, 2-[4′-iodobenzoyl]-oxo-ethyl methacrylate and 2-[2′,3′,5′-triiodobenzoyl]-oxo-ethyl methacrylate. Combinations of more than one radiopaque monomer are also possible.

In a preferred embodiment 2-[4′-iodobenzoyl]-oxo-ethyl methacrylate is used, since this crystalline material can be easily prepared in bulk-quantities in pure form.

In a preferred embodiment 2-[2′,3′,5′-triiodobenzoyl]-oxo-ethyl methacrylate is used, which is useful to introduce a high level of X-ray contrast in the copolymer, since during polymerisation three iodine atoms are introduced per monomer.

A hydrophilic monomer in the context of this invention is meant to be any monomer having a strong affinity for water, tending to dissolve in, mix with, or be wetted by water.

Examples of suitable hydrophilic monomers are, but not limited to, N-vinyl-2-pyrrolidinone, 2-hydroxy ethyl methacrylate, methacrylic acid, polyethylene glycol methacrylate, vinyl alcohol or derivatives thereof. It is important that at least one hydrophilic monomer is used in the method of the invention, but also mixtures of hydrophilic monomers can be used. Preferably, the hydrophilic monomer is 2-hydroxy ethyl methacrylate and/or N-vinyl-2-pyrrolidinone.

The molar ratio between the at least one hydrophilic monomer and the at least one radiopaque monomer can be varied in dependence of specific monomers used and the required level of radio-opacity. The minimum level thereof is determined by the location where the embolisation should take place. If this is very deep into the human body, higher levels are required. The ratio of the radio-opaque monomer to the hydrophilic monomer is thus on the one hand a factor of the level of radio-opacity and on the other hand of the minimal hydrophilicity. A good value for determining this hydrophilicity is the equilibrium amount of swelling in water of 20° C. This percentage is at least 10%, on the basis of the measurement of the volume of the particles. Generally a hydrophilic microsphere according to the invention can imbibe water up to a volume increase of the microsphere of at least 10%. Preferably the volume increase of the microsphere is at least 15%. Most preferably, the volume increase of the microsphere is at least 20%

On the other hand, the I and/or Br-content should at least be 5 wt. %. Generally speaking this will result in a ratio of the two types of monomers, which varies (on the basis of the number of monomeric units), between 1-20 and 20-1 i.e. hydrophilic versus radio-opaque. A preferred range is between 17:1 and 2.5:1 An increase of the radio-opaque monomer content results in a decrease of the water uptake. On the other hand, a decrease of the radiopaque monomer content results in worse X-ray visibility.

It is preferred that the particles are substantially spherical in shape.

The particles of the invention are homogeneous, which means that the radiopaque monomer is evenly distributed over the volume of the sphere i.e. there exists no gradient in the distribution of the radiopaque monomer from the outer to the inner parts of the sphere. A preferred method to obtain spherical particles is the particles are prepared via a suspension polymerisation process. For embolisation, spherical particles allow a simple transcatheteral introduction without aggregation of particles. Furthermore, the spherical particles can better penetrate in the blood vessel and a geometrically better blocking of the vascular lumen compared to non-spherical particles is obtained.

The average particle diameter is at least 10 μm, preferably 10-2000 μm, more preferably 50-1000 μm. It was found that an increase in average particle diameter leads to an increase of the X-ray visibility. For super precise embolisation, however, small particles are required. Nevertheless, the specific use determines the best size and size range.

It is to be noted that the particles are substantially non-porous. The invention resides at least partly therein that contrary to the teachings of, for example, Horak et al, optimal embolisation particles do not need to be porous and are actually non-porous. Due to this, the particles are very well visible in X-Ray, which means that the introduction into the body and the dispersion, respectively localization can be followed very good.

It is also preferred that the iodine content of the particles is 5-60 wt. % based on the dry weight of the particle, more preferably 10-50 wt. %, most preferably 15-40 wt. %. It was found that an increase in iodine content results in an increase of the X-ray visibility.

Because of the hydrophilic nature of the radiopaque particles of the invention, the material is soft and compressible. As a result, the particles of the invention perform better in vascular occlusion than rigid particles.

The invention is also directed to a method for preparing the embolic radio-opaque copolymer particles, comprising the suspension polymerisation of at least one hydrophilic monomer with at least one radio-opaque monomer according to general formula

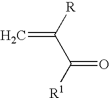

wherein R is H, methyl or ethyl, and R¹ is I, Br or

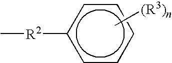

wherein R² is O, NH, O—[CH₂—CH₂—O]ₚ—C(O)—, O—[CH₂]ₘ—O—C(O)—, O—[CH₂]ₚ—, NH—[CH₂—CH₂—O]ₚ—C(O)—, NH—[CH₂]ₘ—O—C(O)—or NH—[CH₂]ₚ— wherein m>1 and p≧1, R³ is I or Br and n is 1, 2 or 3.

The temperature at which the suspension polymerisation is carried out is dependent on the nature of the monomers and the type and amount of initiator. In addition the properties of the polymer produced is influenced also by these factors (temperature, amount and type of initiator). Generally the temperatures ranges between about 50° C. and the boiling point of the polymerisation system at the pressure used. As it is preferred to use ambient pressure, the upper limit will generally be about 95° C. At higher pressures, such as up to 15 bar(abs) temperatures up to 200° C. may be used.

Polymerisation times are dependent on the factors of temperature and type and amount of initiator. It is preferred to continue the polymerisation until the amount of residual monomer is sufficiently low, i.e. at such a level that no appreciable amounts of monomer leach out from the particles. In the alternative it is possible to steam the particles to evaporate residual monomer.

Generally, the polymerisation time is between about 30 min and 24 hours.

After the suspension polymerisation the particles can be isolated, washed and dried for further applications. In order to further narrow the size distribution of the particles it is possible to sieve the dried particles in batches of well-defined sizes. This is particularly advantageous when the particles are used for embolisation.

The suspension polymerisation can be carried out in the presence of a suitable suspension stabiliser, such as for instance magnesium hydroxide, and/or a surface active agent. Further it is preferred that a polymerisation initiator is present. Suitable polymerisation initiators are for instance 2,2′-azobis(isobutyronitrile), dibenzoyl peroxide or tert-butyl peroxybenzoate.

It is also possible to carry out the suspension polymerisation in the presence of a crosslinker. A suitable crosslinker is for example allylmethacrylate. This in particular advantageous for the stability of the spheres; crosslinking prevents that the spheres can dissolve in any solvent. It is to be noted that the particles advantageously should be at least slightly compressible. This is important in order that the particles can function properly in the embolisation, where the compressibility allows the particles to improve the clogging of the vessels. A certain amount of crosslinker can be used to fine tune the compressibility.

The water to monomer ratio is generally in the conventional range, as is know in the art.

Typically, the suspension polymerisation is carried out in a concentrated solution of salt, such as sodium chloride, in water. The presence of salt is important in view of the hydrophilic nature of one of the monomers and the presence of salts keeps these monomers inside the suspended particles and prevents dissolution into the water phase of the polymerization mixture.

The invention is further directed to the use of the radio-opaque copolymer particles of the invention as embolic agent. Most commercially available embolic agents are radiolucent, i.e. they are invisible on X-ray images. These embolic particles are usually dispersed in saline which has been enriched with contrast medium. This has the disadvantage that fluoroscopic exploration, which is performed during injection of the embolic agent through a catheter, only provides information about the location of the fluid and not about the embolic particles. In case of improper dispersion of the particles, it is well possible that the liquid can pass more distal in the tumor than the particles, so improper location of the embolic agent is inferred from this method. Also, sometimes solid materials that are capable of absorbing X-ray radiation, like small metallic particles, are added to the embolic agent. However, for such method there will always be a risk of leakage of the contrast agent, which again precludes exact location of the embolic agent. In contrast, the radiopaque particles of the invention are intrinsically radiopaque and therefore allow an exact location of the embolic material.

Further, the invention is directed to the use of radio-opaque particles according to the invention in the manufacture of a medicament for treating arteriovenous malformations, intracranial meningiomas, neoplasms, fibroids, or tumors.

### Example 1

In a 250 mL round bottom flask, 14.64 g of NaCl and 2 g of MgCl₂.6H₂O were dissolved in 70 mL of distilled water and heated to 75° C. under continuous mechanical stirring. At this temperature, 0.78 g of NaOH dissolved in 15 mL of distilled water was added dropwise to this solution. This resulted in precipitation of Mg(OH)₂, the suspension stabiliser. After complete addition of this solution, the temperature was further raised to 80° C. In a next step, the organic phase, containing 10 wt. % of iodine, was added dropwise to the water phase. The organic phase consisted of 14.32 g of 2-hydroxy ethyl methacrylate, 5.68 g of 2-(4′-iodobenzoyl)-oxo-ethyl methacrylate and 80 mg of 2,2′-azobis(isobutyronitrile). The temperature was then left for 4.5 hours at 80-85° C. During all these steps mechanical stirring was continued. After completion of the reaction, diluted HCl was added to dissolve the stabiliser. Subsequently, the formed spheres were washed several times with distilled water and the product was freeze-dried. The dried spheres were characterised for their size by light microscopy and then they were sieved in batches of well-defined size.

Subsequently, the volume swelling ratio and X-ray visibility of the particles were determined.

### Example 2

In a 250 mL round bottom flask, 14.64 g of NaCl and 2 g of MgCl₂.6H₂O were dissolved in 70 mL of distilled water and heated to 75° C. under continuous mechanical stirring. At this temperature, 0.78 g of NaOH dissolved in 15 mL of distilled water was added dropwise to this solution. This resulted in precipitation of Mg(OH)₂, the suspension stabiliser. After complete addition of this solution, the temperature was further raised to 80° C. In a next step, the organic phase, containing 15 wt. % of iodine, was added dropwise to the water phase. The organic phase consisted of 5.75 g 2-hydroxy ethyl methacrylate, 5.75 g of N-vinyl-2-pyrrolidinone, 8.51 g of 2-(4′-iodobenzoyl)-oxo-ethyl methacrylate and 80 mg of 2,2′-azobis(isobutyronitrile). The temperature was then left for 4.5 hours at 80-85° C. During all these steps mechanical stirring was continued. After completion of the reaction, diluted HCl was added to dissolve the stabiliser. Subsequently, the formed spheres were washed several times with distilled water and the product was freeze-dried. The dried spheres were characterised for their size by light microscopy and then they were sieved in batches of well-defined size.

Subsequently, volume swelling ratio and X-ray visibility of the particles were determined.

### Example 3

In a 100 mL round bottom flask, 2.17 g of NaCl and 0.3 g of MgCl₂.6H₂O were dissolved in 9 mL of distilled water and heated up to 75° C. under continuous magnetic stirring. At this temperature, 0.12 g of NaOH dissolved in 4 mL of distilled water was added dropwise to this solution. This resulted in precipitation of Mg(OH)₂, the suspension stabiliser. After addition of this mixture the temperature was further raised to 80° C. In a next step, the organic phase containing 20 wt. % of iodine, was added dropwise to the water phase. The organic phase consisted of 1.44 g of N-vinyl-2-pyrrolidinone, 0.56 g of 2-hydroxy ethyl methacrylate, 1.00 g of 2-[2′,3′,5′-triiodobenzoyl]-oxo-ethyl methacrylate, 71.6 mg of allylmethacrylate and 14 mg of 2,2′-azobis(isobutyronitrile). The temperature was then left for 5 hours at 80-85° C. and for 20 hours at 50° C. During all these steps mechanical stirring was continued. After completion of the reaction, diluted HCl was added to dissolve the stabiliser. Subsequently, the formed spheres were washed several times with distilled water and the product is freeze dried. The dried spheres are characterised for their size by light microscopy and then they are sieved in batches of well-defined size.

Subsequently, the volume swelling ratio and X-ray visibility have been determined.

TABLE 1
Average particle size and volume swelling ratio of the formed particles.

| | Average sphere size | Volume swelling ratio |
|---|---|---|
| Example 1 | 314 ± 109 | 1.28 |
| Example 2 | 286 ± 127 | 1.32 |
| Example 3 | 366 ± 92 | 1.10 |

1. Embolic material comprising spherical, homogeneous and substantially non-porous radiopaque polymer particles based on at least one hydrophilic monomer and at least one radiopaque monomer according to general formula 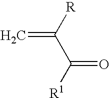
wherein R is H, methyl or ethyl, and R¹ is I, Br or 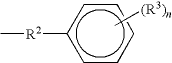
wherein R² is O, NH, O—[CH₂—CH₂—O]ₚ—C(O)—, O—[CH₂]ₘ—O—C(O)—, O—[CH₂]ₚ—, NH—[CH₂—CH₂—O]ₚ—C(O)—, NH—[CH₂]ₘ—O—C(O)— or NH—[CH₂]ₚ— wherein m>1 and p≧1, R³ is I or Br and n is 1, 2 or 3,
the iodine and/or bromine content being at least 5 wt. % based on the dry weight of the particle,
the said particles having an average particle diameter of at least 10 μm and being able to imbibe water up to a volume increase of the particle of at least 10%.

2. Radiopaque copolymer particles according to claim 1, wherein m is 2 and p is 1 or 2.

3. Embolic material according to claim 1, wherein the at least one radiopaque monomer is chosen from the group consisting of 2-[2′-iodobenzoyl]-oxo-ethyl methacrylate, 2-(4′-iodobenzoyl)-oxo-ethyl methacrylate and 2-[2′,3′,5′-triiodobenzoyl]-oxo-ethyl methacrylate.

4. Embolic material according to claim 1, wherein the at least one hydrophilic monomer is chosen from the group consisting of N-vinyl-2-pyrrolidinone, 2-hydroxy ethyl methacrylate, methacrylic acid, polyethylene glycol methacrylate, vinyl acetate as a precursor for vinyl alcohol or derivatives thereof.

5. Embolic material according to claim 1, wherein the particles have an average diameter of 10-2000 μm.

6. Embolic material of claim 1, having an iodine content of 5-60 wt. % based on the dry weight of the particle.

7. Embolic material according to claim 1 for use as a medicament.

8. Method for preparing radiopaque embolic copolymer particles comprising the suspension polymerisation of at least one hydrophilic monomer with at least one radiopaque monomer according to general formula 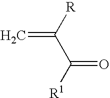
wherein R is H, methyl or ethyl, and R¹ is 1, Br or 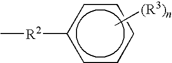
wherein R² is O, NH, O—[CH₂—CH₂—O]ₚ—C(O)—, O—[CH₂]ₘ—O—C(O)—, O—[CH₂]^{p}—, NH—[CH₂—CH₂—O]^{p}—C(O)—, NH—[CH₂]ₘ—O—C(O)—or NH—[CH₂]ₚ— wherein m>1 and p≧1, R³ is I or Br and n is 1, 2 or 3.

9. The method of claim 8, wherein the suspension polymerisation is carried out in the presence of a methacrylate or dimethacrylate crosslinker.

10. Use of radiopaque copolymer particles according to claim 1 as an embolic agent.

11. Use of at least one hydrophilic monomer and at least one radiopaque monomer according to general formula 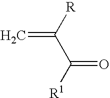
wherein R is H, methyl or ethyl, and R¹ is I, Br or 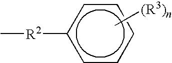
wherein R² is O, NH, O—[CH₂—CH₂—O]ₚ—C(O)—, O—[CH₂]ₘ—O—C(O)—, O—[CH₂]ₚ—, NH—[CH₂—CH₂—O]^{p}—C(O)—, NH—[CH₂]ₘ—O—C(O)—or NH—[CH₂]ₚ— wherein m>1 and p≧, R³ is I or Br and n is 1, 2 or 3 in the manufacture of a radiopaque copolymer particle for treating arteriovenous malformations, intracranial meningiomas, fibroids, neoplasms, or definitive treatment of tumors.
